(19) United States
(12) Patent Application Publication
(10) Pub. No.: US 2013/0178378 A1
(43) Pub. Date: Jul. 11, 2013

(54) MULTIPLEX DIGITAL PCR

(71) Applicants: Andrew C. HATCH, Queen Creed, AZ (US); Abraham LEE, Irvine, CA (US)

(72) Inventors: Andrew C. HATCH, Queen Creed, AZ (US); Abraham LEE, Irvine, CA (US)

(21) Appl. No.: 13/492,698

(22) Filed: Jun. 8, 2012

Related U.S. Application Data
(60) Provisional application No. 61/495,308, filed on Jun. 9, 2011.

(51) Int. Cl. C12Q 1/68

(52) U.S. Cl. CPC C12Q 1/686
USPC 506/9; 506/16

(57) ABSTRACT

Embodiments of the claimed subject matter are directed to the ability to further multiplex in the digital regime using combinatorial color, temporal, and intensity encoding of probe sequences for a greater number of total signal readouts. A digital PCR solution is provide which enables the unique ability to identify a greater number of fluorescent probe sequences by using multiple color, temporal, and intensity combinations to encode each unique probe sequence. Furthermore, less expensive real-time PCR amplification indicators such as PicoGreen can be used to achieve multiplexed digital PCR based on temporal cues, intensity cues, or intensity and temporal cues combined, thus distinguishing primer pairs at greater degrees with significant cost reductions. These can also be used to enhance controls and normalize results for greater accuracy.

### CLAIM OF PRIORITY

This application claims priority to U.S. Provisional Patent Application Ser. No. 61/495308, filed Jun. 9, 2011, and which is hereby incorporated by reference.

### ACKNOWLEDGEMENT OF GOVERNMENT SUPPORT

This invention was made with Government support under Grant no. HR0011-06-1-0050, awarded by the DARPA. The Government has certain rights in this invention.

### TECHNICAL BACKGROUND

Polymerase chain reaction (PCR) is a technique used in molecular biology to amplify one or more instances of a sequence or segment of DNA across several orders of magnitude, generating millions of copies of a particular DNA sequence. PCR is now a common and often indispensable technique used in medical and biological research labs for a variety of applications.

Typically, PCR methods employ thermal cycling, i.e., alternately heating and cooling of the reaction for DNA melting and enzymatic replication of the DNA. Primers (DNA oligonucleotides) containing sequences complementary to the target region along with a DNA polymerase (such as a Taq polymerase) are key components in the reaction to enable selective and repeated amplification. As a PCR process progresses, the DNA generated is itself used as a template for replication, setting in motion a chain reaction in which the DNA template is exponentially amplified.

Real-time PCR (qPCR) is a technique based on PCR, and used to amplify and simultaneously quantify a targeted DNA molecule. For one or more specific sequences in a DNA sample, Real Time-PCR enables both detection and quantification. A real-time PCR procedure follows the general principle of polymerase chain reaction; its key feature being that the amplified DNA is detected as the reaction progresses in real time. Typically, qPCR processes require one of two components for detecting amplified DNA sequences in real-time PCR. (1) non-specific fluorescent dyes that intercalate with any double-stranded DNA, or (2) sequence-specific DNA probes consisting of oligonucleotides that are labeled with a fluorescent reporter which permits detection only after hybridization of the probe with its complementary DNA target 5′ exonuclease activity of the enzyme to cleave the quenched probe from the primer releasing it into the bulk solution where its fluorescence intensity increases.

Digital PCR (dPCR) overcomes the difficulties of conventional PCR processes. With dPCR, a sample is partitioned so that individual nucleic acid molecules within the sample are localized and concentrated within many separate regions. The partitioning of the sample allows the molecules to be counted by estimating according to a Poisson distribution. As a result, each part will contain “0” or “1” molecules, or a negative or positive reaction, respectively. After PCR amplification, nucleic acids may be quantified by counting the regions that contain PCR end-product, positive reactions. In conventional PCR, the starting copy number is proportionally quantified to the number of PCR amplification cycles required to reach a threshold fluorescence intensity. Digital PCR, however, is not dependent on the number of amplification cycles to determine the initial sample amount, eliminating the reliance on uncertain exponential data to quantify target nucleic acids and providing absolute quantification.

Multiplex polymerase chain reaction (Multiplex PCR) is another modification of polymerase chain reaction and is used in order to rapidly detect multiple gene sequences in a single PCR reaction. Multiplex PCR is typically accomplished using multiple primer sequences, each with a unique fluorophore for detection and quantification. This process amplifies DNA samples using the primers along with temperature-mediated DNA polymerases in a thermal cycler. Multiplex-PCR consists of multiple primer sets within a single PCR mixture to produce amplicons that are specific to different DNA sequences.

Typically, as much as 5-plex real-time qPCR is achievable in a PCR mixture by using fluorescentty labeled probes, each one corresponding to a unique DNA sequence, which when amplified by a DNA polymerase, emit a fluorescence signal at its specified spectral wavelength. The spectral frequency discrimination between different fluorophores, or reporters, attached to each probe sequence enables detection of up to five different amplicon sequences, one for each fluorescent color that can be identified. Multiplexing beyond 5-plex is difficult due to insufficient spectral wavelengths that can be optically distinguished using current state of the art fluorescence excitation and emission filter sets. Furthermore, Multiplex PCR reactions suffer from high degrees of competition for limited resources making it difficult to achieve high levels of multiplexing.

In addition, no more than a single probe sequence can be used per spectral wavelength to further increase multiplexing ability, because one would be unable to determine what percentage of the total PCR amplified fluorescence intensity at that wavelength corresponds to more than a single probe sequence. For example, if two probes, A and B, were labeled with the same fluorescent marker, green, one wouldn't know if the amplified green signal was a composition of 100% probe A, 100% probe B, or some mixture of the two. For this same reason, multiplexing cannot be achieved using non-specific reporters such as intercalating dyes because one cannot differentiate amplified signal arising from any more than a single PCR primer pair. This is because these dyes report all double-stranded nucleotide strands with no distinction among sequence aside from the primer pairs that select for amplification. On the other hand, specific reporters such as DNA probes with quenchers are expensive, and increasing the number of different fluorescent reporters (e.g., for high-degree multiplexing) can quickly become cost-prohibitive.

### SUMMARY

This Summary is provided to introduce a selection of concepts in a simplified form that is further described below in the Detailed Description. This Summary is not intended to identify key features or essential features of the claimed subject matter, nor is it intended to be used to limit the scope of the claimed subject matter.

To overcome the problems noted above, a Digital PCR (dPCR) solution is provided as an alternate method to qPCR, and which improves on existing dPCR solutions. A dPCR process involves discretizing a larger PCR volume into several smaller reaction volumes, and limiting the number of DNA amplicons in the PCR solution to favor no more than a single amplicon per reaction volume, thus each reactor will have a 0 or 1 output. A Poisson distribution is used to predict the digital regime where only a single DNA amplicon will occur in a randomly discretized volume reactor to favor only one DNA amplicon of interest per reaction volume. In this way, the PCR amplified signal (e.g., a fluorescence) emitted by each reactor volume is the product of only one amplicon and is isolated from all other discrete reactor volumes. Quantification is then achieved by counting how many digital reactors emit an amplified fluorescent signal corresponding to an intercalating dye or a particular DNA polymerase probe sequence. Since each reactor volume is limited to no more than a single DNA strand in the digital regime, one can correctly assume that 100% of its amplified fluorescence signal comes from only that one DNA strand and corresponding primer and probe set.

Embodiments of the claimed subject matter are directed to the ability to further multiplex beyond conventional methods in the digital regime using combinatorial color, temporal, and intensity encoding of probe sequences for a greater number of total signal readouts. In an embodiment, a digital PCR method is provided that enables the unique ability to identify a greater number of fluorescent probe sequences (e.g., TaqMan probe sequences) by using multiple color, temporal, and intensity combinations to encode each unique probe sequence. Furthermore, less expensive non TaqMan-probe real-time PCR amplification indicators such as SYBR- or PicoGreen can be used to achieve multiplexed digital PCR based on temporal cues alone, intensity cues alone, or intensity and temporal cues combined, thus distinguishing primer pairs at greater degrees with significant cost reductions. These can also be used to enhance controls and normalize results for greater accuracy if desired. Using this concept one can increase the typical multiplexing limits from typical 5-plex qPCR to as much as 100-plex digital PCR with limited spectral bands using fluorescent reporters. This approach has several financial and capability based improvements to current state of the art multiplexing approaches.

According to embodiments of the claimed subject matter, previous limits for multiplexing are exceeded with digital PCR by ensuring no more than a single DNA strand per reactor volume to multiplex more colors, and combine more intensity profiles per probe and still ensure that the signal obtained correlates to no more than a single DNA strand. In still further embodiments, the technology also provides the use of low cost non-specific fluorescence reporters like PicoGreen to enable multiplex digital PCR quantification. In an embodiment, 100-plex digital PCR or more is achieved using combinatorial encoding of color and intensity, as compared to only 5-plex qPCR. Further advantages provided by embodiments of the claim subject matter include reduced cost schemes, internal controls and lower background noises for greater multiplexing by using a variety of probe distinguishing cues in the temporal, spectral, and intensity domains.

Applications of various aspects of the presently claimed subject matter may include detecting diseases or performing rapid genotyping of DNA solutions in low concentrations for rare detection and extremely high multiplex capabilities. Further applications could be used for medical, military, or environmental screening among other things.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 depicts an illustration of an exemplary system for digital PCR multiplexing, in accordance with embodiments of the present disclosure.

FIG. 2 depicts a data flow diagram of a process for digital PCR multiplexing, in accordance with embodiments of the present disclosure .

FIG. 3 depicts an illustration of multiplex digital PCR primer/probe differentiation using intensity encoding of TaqMan probes in limited concentrations, in accordance with embodiments of the present disclosure.

FIG. 4 depicts an illustration of multiplex digital PCR primer set differentiation using intensity encoding of intercalating dyes in primer limited concentrations, in accordance with embodiments of the present disclosure.

FIG. 5 depicts an illustration of temporal distinctions between TaqMan primer/probe pairs to encode different probes using a single color spectrum, in accordance with embodiments of the present disclosure

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known processes, procedures, components, and circuits have not been described in detail as not to unnecessarily obscure aspects and features of the subject matter.

Specifically, portions of the detailed description that follow are presented and discussed as mathematical formulas. These formulas are provided for illustrative purposes, and it is understood that the claimed subject matter is not limited to these formulas, and that embodiments are well suited to alternate expression.

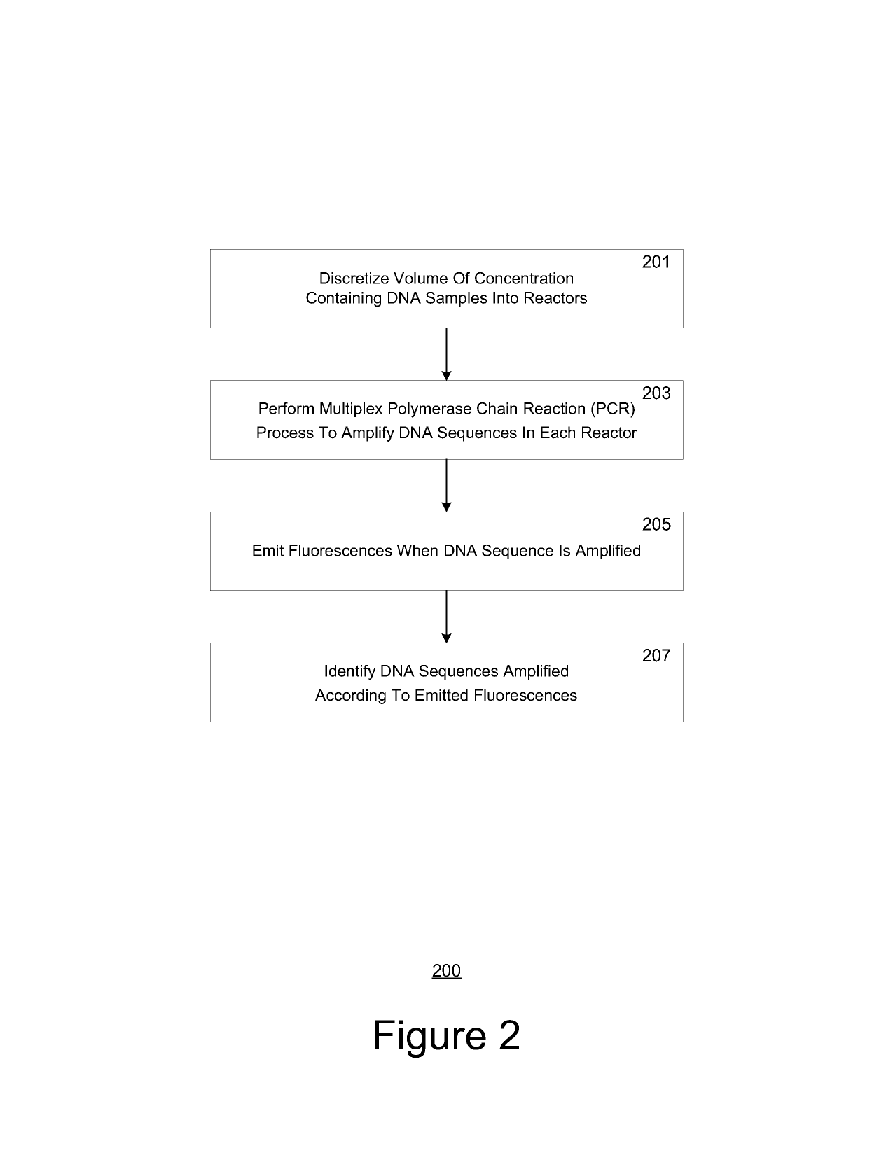

Portions of the detailed description that follow are also presented and discussed in terms of a process. Although steps and sequencing thereof are disclosed in figures herein (e.g., FIG. 2) describing the operations of this process, such steps and sequencing are exemplary. Embodiments are well suited to performing various other steps or variations of the steps recited in the flowchart of the figure herein, and in a sequence other than that depicted and described herein.

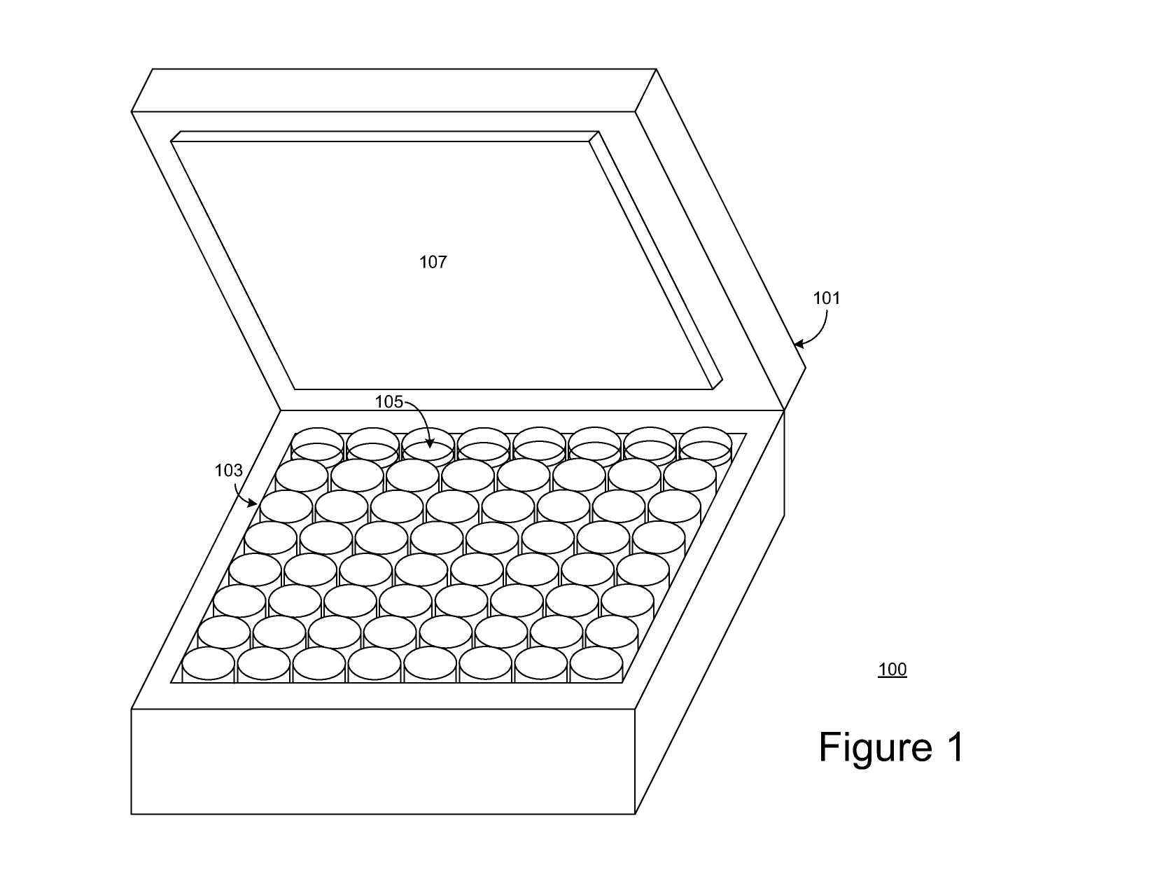

As depicted in FIG. 1, an illustration of an exemplary system 100 for digital PCR multiplexing is presented in accordance with embodiments of the present disclosure. As presented in FIG. 1, the system 100 includes a device for performing PCR, such as a thermal cycler 101. The thermal cycler is further equipped with a plurality of reactors 103 and a heating element 107 for adjusting the temperature in the plurality of reactors 103, such as a multiplex assay. In an embodiment, the reactors 103 may contain a volume of concentration 105 which includes discretized droplets each containing a digitized amount (e.g., 0 or 1) of a DNA strand. Alternately, the droplets can also function as a discrete reactor—without the need for further distribution into separate dedicated containment units. In still further embodiments, the droplets may further contain components for PCR and PCR multiplexing, such as preliminaries, nucleotides, primers and fluorescent reporters. Fluorescent reporters may be implemented as intercalating dyes (e.g., PicoGreen) or fluorescently labeled probes.

In an embodiment, the end-point fluorescence of a small reactor volume can be detected as a single 0 or 1 output with each positively fluorescing reaction volume directly corresponding to a single DNA strand. By counting the number of positive vs. negative fluorescing volume reactions, the output of the initial DNA template concentration may be obtained. According to various embodiments, by leveraging multiplex solutions with probe distinguishing using color combination cues, fluorescence intensity cues, or temporal fluorescence cues, or a combination of these cues, advanced multiplexing can be achieved with the system 100. In further embodiments, the system 100 can achieve 100-plex digital PCR or more with a sufficient number of discrete reactors, as compared to only 5-plex qPCR using combinatorial encoding of color and intensity.

As depicted in FIG. 2, a data flow diagram of a process 200 for digital PCR multiplexing is presented, in accordance with embodiments of the present disclosure. Steps 201 to 207 describe exemplary steps comprising the process 200 depicted in FIG. 2 in accordance with the various embodiments herein described.

At step 201, a volume of concentration containing DNA samples is discretized into individual reactors (e.g. droplets). In an embodiment, the volume is discretized into droplets, which are subsequently distributed into several smaller reaction volumes contained in reactors (e.g., reactors 103), such that the number of amplicons (e.g., DNA strands) in the PCR solution is limited to no more than a single amplicon per reaction volume, on average, and thus each reactor will have a 0 or 1 output for a PCR process. In alternate embodiments, the droplets themselves can be used as reactors, and the distribution into dedicated reactors may be omitted. According to some embodiments, a Poisson distribution may be used to predict the digital regime where only a single DNA amplicon will occur in a randomly discretized volume reactor to favor only one DNA amplicon of interest per reaction volume. Alternate techniques may also be implemented for distribution. Discretizing the volume of concentration into droplets may be achieved by a binary splitting apparatus (not shown), wherein a single source traverses a plurality of bifurcated junctions until eventually the volume becomes distributed in discretized units (droplets).

At step 203, a PCR process is performed to detect and/or amplify DNA sequences from the strands of DNA in the plurality of reactors. A typical PCR process includes administering a plurality of thermocycles to the reactors to perform a plurality of steps. These steps may include, for example, an initialization step, a denaturation step, an annealing step, and finally, an extension step whereupon, given sufficient reagents, the amount of the DNA sequences targeted by the primers is increased (amplified).

At step 205, fluorescence is emitted during the course of DNA amplification occurring in step 203. The fluorescence is emitted by fluorescent reporters (e.g., a intercalating dye such as SYBR or PicoGreen, or fluorescent probes) contained in the volume of concentration. In an embodiment, the fluorescent probe may be implemented as a TaqMan probe. TaqMan probes are hydrolysis probes that are designed to increase the specificity of PCR assays. A standard TaqMan probe comprises a fluorophore covalently attached to the 5′-end of an oligonucleotide probe and a quencher at the 3′-end. During PCR amplification, the primers and fluorescently tagged probes anneal to the DNA template, and as the polymerase extends the primer sequences, the fluorescent label is cleaved from the probe strand, thereby increasing its distance from the quencher and allowing the fluorophore to emit fluorescence with greater intensity. Intercalating dyes, on the other hand, bind to double-stranded DNA sequences, and an increase in DNA product during PCR therefore leads to an increase in fluorescence intensity. Fluorescence from a fluorescent reporter is emitted in a pre-defined spectral wavelength (e.g., color).

Finally, at step 207, the fluorescences emitted at step 205 are detected (e.g., via digital filters) and identified, and the DNA sequences corresponding to the emitted fluorescences may be similarly identified based on their correspondence. According to various aspects of the presently claimed subject matter, a single primer may be equipped with multiple reporters that will produce fluorescence in a combination of colors when the DNA sequence corresponding to the primer is amplified. Further aspects provide that fluorescence of varying intensities, or at varying temporal cues, may also be mapped to, and used to identify DNA sequences for additional multiplexing ability. Each of these aspects are described in greater detail below.

DIGITAL PCR MULTIPLEXING WITH INCREASED COLOR ENCODING

According to one aspect, fluorescent probes that emit a combination of colors to correctly identify different sequences may be implemented for increased color-coded multiplexing. For example, using 2 separate colors, red and green, three separate probe sequences may be encoded as follows:
Probe 1—red
Probe 2—green
Probe 3—red and green

Further increasing this to 3 colors: red, green, and blue, the following is achieved:
Probe 1—red
Probe 2—green
Probe 3—blue
Probe 4—red and green
Probe 5—green and blue
Probe 6—red and blue
Probe 7—red, green, and blue

Thus, the number of n separate color combinations enables (n!+1) total color combinations for n>1. For n=2, 3, 4 or 5 color multiplex systems the following combinations may be derived:
2!+1=3 color combinations
3!+1=7 color combinations
4!+1=25 color combinations
5!+1=121 color combinations

Color combinations can be pre-determined by associating a single primer with two (or more) colors at known concentrations, and mapping the data to target DNA sequences. Other embodiments which will be discussed in greater detail in subsequent paragraphs to include intensity and temporal cues. By limiting the number of DNA strands in each reactor to at most 1, any fluorescent signal emitted from the droplet can only be the result of the DNA strand. By also limiting the dilution (e.g., by limiting the concentration of the fluorescent reporter), combinations of similar colors may be distinguished due to varying intensities of the colors produced. Thus, in the tri-color example described above, a reactor that amplifies with primers corresponding to Probes 1 (100% red), 2 (100% green), and 3 (100% blue) will produce the color combination of red, green, and blue with a greater intensity than the color combination produced corresponding to Probe 7 (33% red, 33% green, and 33% blue) due to the higher concentration of fluorescent reporters. According to these implementations, multiplexing to identify the corresponding DNA sequence matching specific color combinations or single color intensities can be increased over conventional methods.

Furthermore, the threshold of what amplified signal intensity is reached, and the cycle number of when that threshold occurs, if at all, can further be tuned in such a way that discrete intensity and temporal encodings are provided for an even greater number of probe sequences per fluorescent reporter. During PCR amplification, the number of total amplified DNA strands per n thermocycles increases at an optimal efficiency of 2ⁿ strands per cycle and consumes fluorescent probes at a similar, although not identical, rate. At lower efficiencies, E<100%, the amplification of DNA using PCR occurs at a rate of (1+E)ⁿ. Thus fluorescence amplification of reporter probes have both a temporal signature as well as an overall intensity signature which can be distinguished from one another by selecting the end-point cycle number at which end-point detection occurs, or by performing real-time observation of TaqMan probe amplification, to determine temporal differences among probe sequences.

### Digital PCR Multiplexing With Intensity Cues

The following examples will treat concepts relating to how intensity and cycle threshold information can be used to detect multiple primer/probe sequences using the same fluorescent reporter.

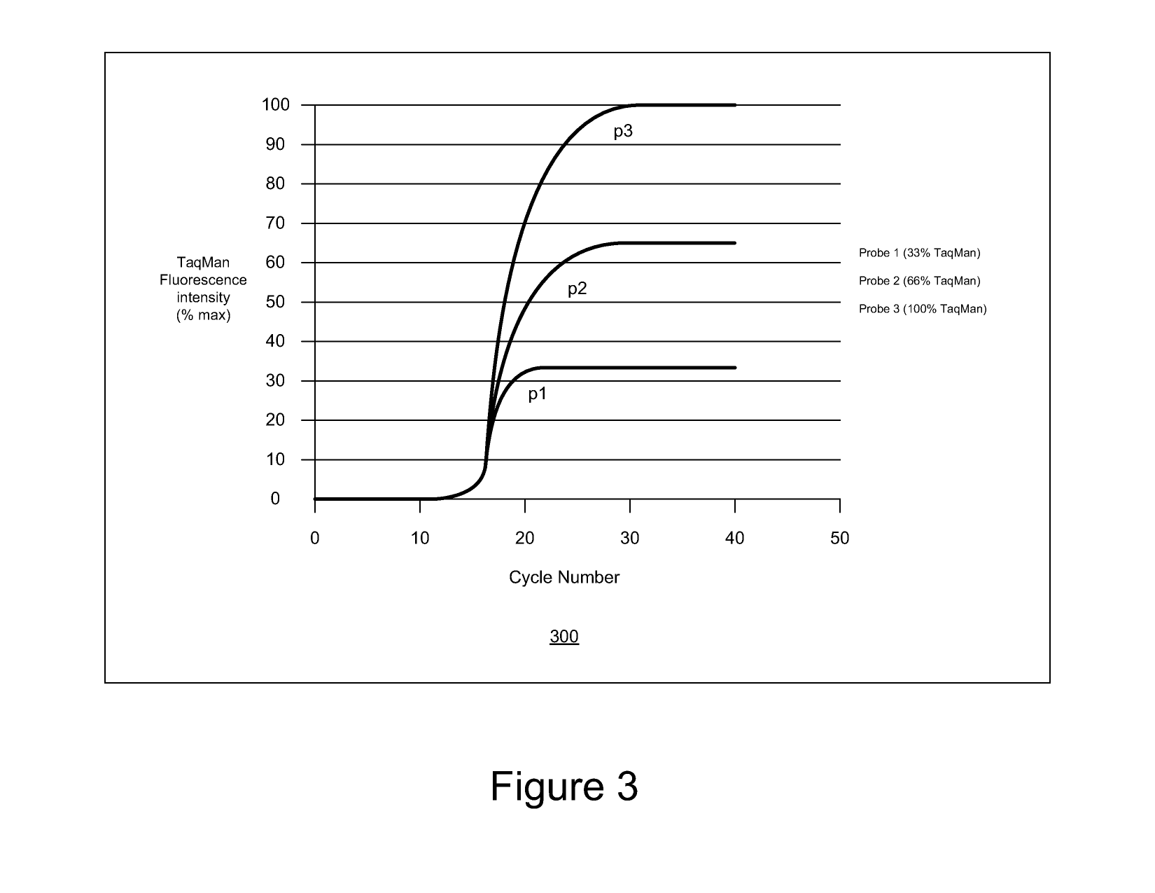

One straightforward approach is to vary the concentration, or total number of probes, which are added to the PCR reagent to encode different intensity levels at complete end-point amplification. FIG. 3 illustrates how this approach is used. In this sense, the cycle number is chosen to be well after the point when complete probe depletion occurs, and no further change in intensity will take place with continued cycle numbers, otherwise known as the end-point intensity level. Alternatively, a real-time observation approach can be used to determine at what cycle numbers the PCR amplification process reached completion and the fluorescence intensity plateaued. This real-time approach gives a greater level of distinction among the processes occurring within each digital reactor as it utilizes the entire temporal envelope. Thus, the final intensity achieved may be distinguished from, how and when it reached that intensity to further differentiate probe sequences for the respective reporters used. This approach requires that the concentration of probes present in the reactor volumes be the limiting reagents for all but the highest intensity level, and be completely consumed, at which point their final fluorescence intensity will correspond to the limited number of probes added to the PCR volume.

For example, using a single fluorescent marker color (e.g., red) three unique probe sequences can be added (e.g., p1, p2, and p3), each labeled with the same color but added to the PCR volume at different concentrations, c1, c2, and c3. The highest concentration c1 corresponds to probe p1 and may be defined as providing 100% signal intensity after reaction completion. Likewise, p2 corresponds to c2 (66%) concentration and p3 to c3 (33%) concentration. Each probe will be depleted at about the same rate until the probes approach depletion near their cycle threshold, at which time the probes will slow down and eventually stop, yielding a total number of unquenched fluorophores directly corresponding to their initial concentration. Running all PCR reactor volumes to total completion (e.g., 45+ cycles), all the probes will be completely amplified to their final end-point fluorescence intensities. Measuring the intensity profile of each digital reactor volume will yield 100% intensity corresponding to P1, 66% intensity corresponding to P2, and all digital reactors expressing 33% maximum intensity will correspond to P3.

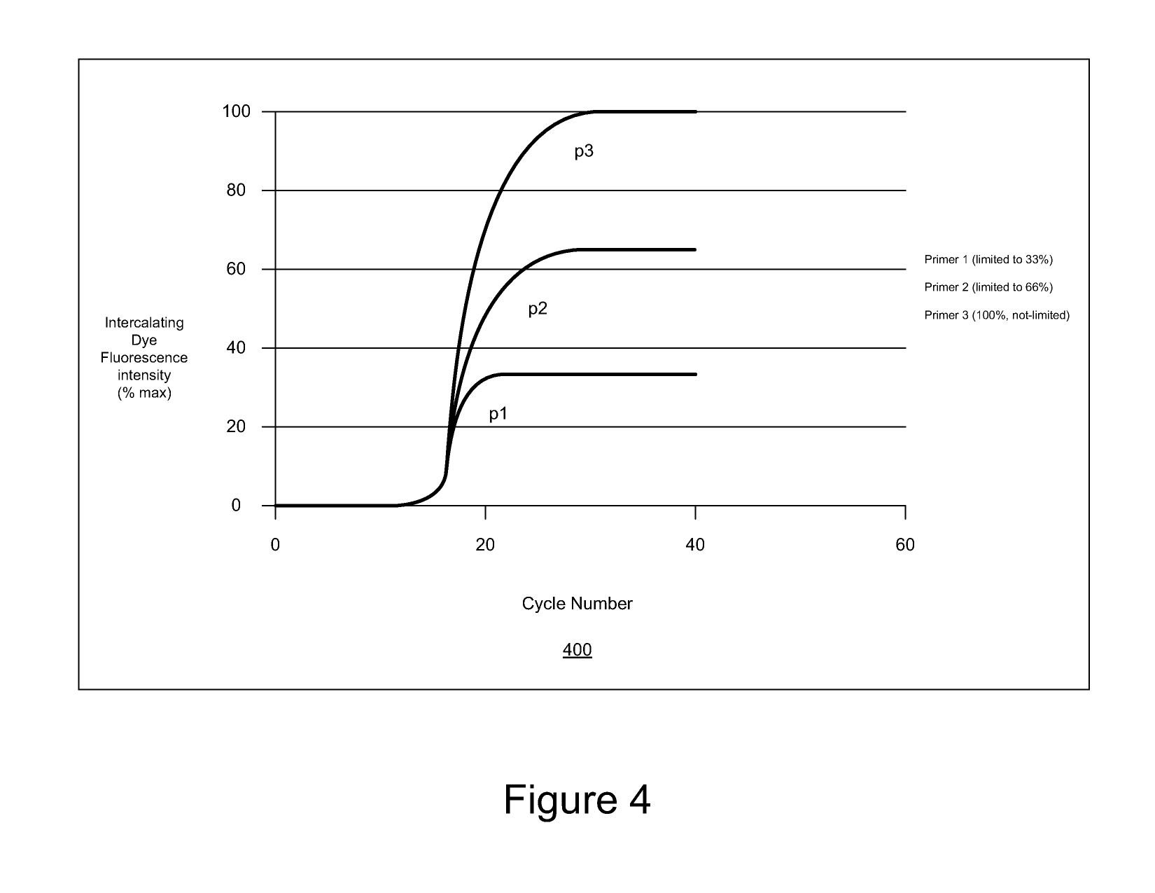

In alternative embodiments, the ability to multiplex is now possible using non TaqMan probe reporters such as intercalating dyes by encoding intensity levels to distinguish concentration limited primer pairs alone. These are advantageous as they are labeled with inexpensive intercalating dyes like SYBR or PicoGreen which bind to amplified double stranded DNA without the need for expensive TaqMan probe reporters. This is done by adding multiple unique primer pairs at different limiting concentrations to yield varying end point fluorescence intensities. FIG. 4 depicts an illustration of multiplex digital PCR primer set differentiation using intensity encoding of intercalating dyes in primer limited concentrations. As depicted in FIG. 4, primer 1 is added at 33% fluorescence intensity, primer 2 is added at 66% fluorescence intensity, and primer 3, at 100% maximal fluorescence intensity. Each primer set will be depleted at about the same rate until they approach their limited threshold at which point, amplification will slow down until the primer reagents are completely consumed. As such, complex TaqMan probes are no longer required for multiplexing, rather, differentiation between the three different primer sequences is based on intensity levels alone, making valuable use of less expensive primer sequences and intercalating dye reporters.

In still further embodiments, the combination of color and intensity cues may be utilized to yield even greater flexibility. According to these embodiments 100-plex or more combinations can be achieved using only three or four color combinations by varying the total concentration and thus final intensity of each digital reactor. For example, using just red and green with 100%, 66%, and 33% intensities results in the following:
Probes 1, 2, 3=red at 100%, 66%, 33%
Probes 4, 5, 6=green at 100%, 66%, 33%
Probes 7, 8, 9=red at 100% and green at 100%, 66%, 33%
Probes 10, 11, 12=red at 66% and green at 100%, 66%, 33%
Probes 13, 14, 15=red at 33% and green at 100%, 66%, 33%

In sum, 15-plex digital PCR is possible with only two color probes. Probe quenchers with higher efficiencies further enhance the multiplexing ability of this ability by enabling lower background signals yielding sufficient signal to noise ratios despite higher probe concentrations of the same fluorophore added to the reactor. In still further embodiments, the use of intercalating dyes may be used as a spectral band for differentiation and detection of specific primers alone, or as a control to indicate how much total DNA has been amplified within each digital reactor. This can then be compared to the intensity bands of the specific reporter labeled probes, to increase specificity and accuracy of the results.

### Digital PCR Multiplexing With Temporal Cues

According to another aspect of the claimed subject matter, multiplexing ability is expanded with temporal signatures by delaying signal amplification, becoming even more versatile, efficient, and cost effective from a reagent perspective. In an embodiment, this technique may be utilized with real-time analysis to determine temporal cues in the PCR amplification process. This technique further allows for increased flexibility in performance and selectivity when considering cycle thresholding applications. Varying methods for delaying signal amplification may be employed, according to various embodiments. In an embodiment, competition during the reaction may be used as a selected method for delaying signal amplification. Other examples of methods to delay signal amplification include, but not are not limited to: primer/probe design, dynamic annealing temperatures during thermocycling, PCR efficiency control, limiting reagents, type of reporter used for detection, and so forth, as described below in greater detail. In an embodiment, TaqMan reporters may be used as fluorescent reporters. Alternately, non-specific intercalating dyes like PicoGreen and others may be used. This is particularly useful to differentiate primer/probe sequences as the reliability and accuracy of detection may be improved, allowing for intensity normalization and internal controls of total PCR amplification as will be described later. Some specific examples of these ideas are given below:

### Competition Based Temporal Shift Using a Single Reporter Color and Non-Labeled Primers

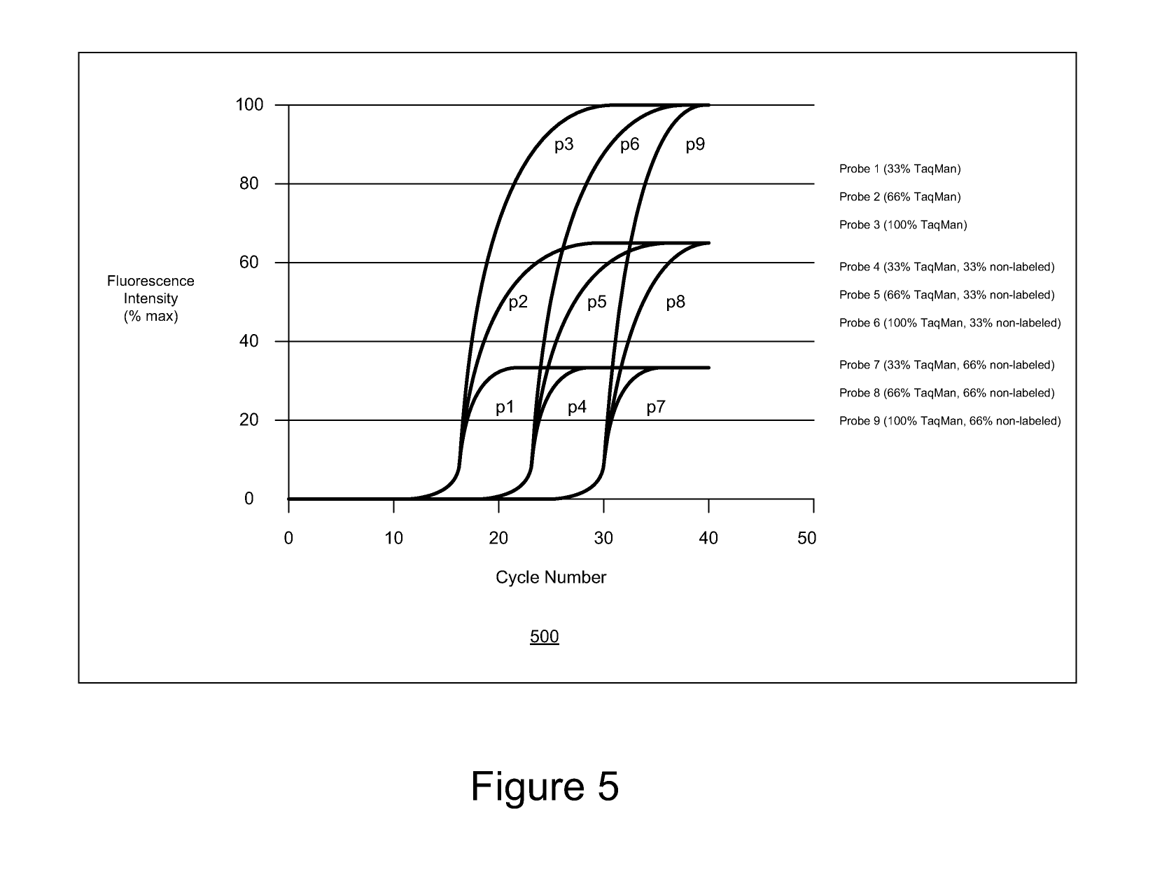

FIG. 5 depicts an illustration of temporal distinction between TaqMan primer/probe pairs to encode nine different probes using a single color spectrum. As depicted in FIG. 5, nine primer/probe sequences p1-p9, all using the same reporter color (e.g., green) are added to a PCR mixture to encode for nine unique amplicons. The sequences consist of probe labeled reporters at 33%, 66%, and 100% concentrations combined with non-labeled probes at varying concentrations to provide temporal shifts in amplification rates. The end-point fluorescence levels of several probe pairs are the same, but the real-time temporal information increases multiplexing ability.

For exemplary purposes, assuming 1×10⁶ amplified probe reporters is the limit of detection for determining the presence of a probe, and assuming the PCR reaction can amplify as much as 1×10⁹ total probes within the digital reactor before reaching the next limiting reagent, with perfect efficiency, 1×10⁶ copies will be consumed at n=20 cycles, and 1×10⁹ copies will be consumed after 30 cycles. At 100% efficiency rate, the amplicon strands will amplify at a rate of 2ⁿ copies for n cycles. If three probe sequences, p1-p3, are added at three different concentrations, say, 33%, 66%, and 100% maximum intensity, all of which are labeled with a green reporter, the probe sequences will be the limiting reagent and amplification will arrest at n=20 cycles. If additional set of three probes sequences is then added (e.g., p4-p6) with the same concentrations of green labeled reporters, in addition to 1×10⁷ non labeled probes, the fluorescence intensity will increase at a slower rate, and the end-point cycle threshold will shift to a later cycle number. With this effect, amplification will be arrested at cycle 25 and the final concentration will still be 33%, 66%, and 100% respectively. Subsequently adding a third set of three probe sequence, p7-p9, with the same concentrations of green labeled reporters and 1×10⁸ non labeled probes, the final fluorescence intensity will increase slower still and shift the cycle number thresholds to even later cycle numbers, e.g., n=30.

According to this embodiment, the concentration levels of reporters added is reduced, thus reducing the cost by using less expensive non-labeled probes, and increased multiplexing ability by several fold. Further, by combining this approach with probes added at higher concentrations and non-labeled probe combinations even higher degrees of multiplexing in a single color band may be achieved. In still further embodiments, intercalating dyes may be added to provide information relating to total PCR amplification, and compared with the TaqMan probe amplified fluorescence intensity, which provides internal controls to correct for errors (e.g. variations in PCR efficiency), and allows even greater levels of distinction among primer probe sequences. For example, not only is there a temporal cue for PCR intensity amplification of the TaqMan labeled probes, but also for the total amplified DNA content within the droplet to allow even greater accuracy and degrees of distinction between primer/probe sequences. This results in a reduction of errors and increases the number of discrete concentration and intensity profiles used in a given color band. Thus, instead of only four concentration levels, one could readily distinguish among five or six levels with less error.

### PCR Efficiency Based Temporal Shift Using Same Fluorescence Intensity and Reporter

Multiplexing ability can also be increased by modifying temporal behavior of signal intensity changes. PCR efficiencies occur at different rates based on a variety of factors, including, but not limited to: primer probe length; specific sequence; how many mismatches there are; annealing temperatures; and additives which may stabilize or destabilize TaqMan polymerase, annealing, or DNA folding. Here we would like to emphasize changes which are specific to the primer and probe designs themselves and not to TaqMan polymerase and amplification efficiency in general. For example, if a primer sequence, pp1, is implemented to anneal at 70 C. and below, a second sequence, pp2, that anneals poorly at 65 C. but anneals well at 60 C. and below, and a third, pp3, that anneals poorly at 60 C. but anneals well at 55 C. and below, three different polymerization efficiencies based on the primer pair sequences alone is achieved. If the annealing temperatures are started out at a higher temperature for the first five or ten cycles, say 65 C., then reduced to 60 C. for the remaining cycles, three different temporal cues may be achieved to differentiate primer pairs.

Similarly, if there is a sufficient difference in efficiency at the same temperature, resulting either from DNA sequence content, or the presence of mismatches in the primer/probe sequences, the annealing temperatures can all remain at 60 C. the entire time but will amplify at different (1+E)ⁿ outcomes allowing for multiplexed detection of different primer/probe pairs. Otherwise, mixed ratios of primers which bind to the same amplicon sequenced regions but have different annealing temperatures can be added to quickly boost an efficiency over the first 10 cycles with a high efficiency primer sequence but which is then depleted and the lower performing primers finish the remainder of the amplification at a reduced efficiency adding another dimension to the temporal envelope of each primer's behavior.

Another aspect is to implement high performing primers, but to use probes which bind with poor efficiency. By combining detection of total amplified DNA levels using an intercalating dye, then detecting and analyzing the amplification efficiency of the probe reporters, another temporal cue is achieved to differentiate different primer/probe pairs. This embodiment is notable because the addition of high concentrations of reporters at the same wavelength increases background noise excessively. By now combining highly specific and selective TaqMan probe based reporters, unique to a single probe sequence, with less selective intercalating dyes which provide secondary information for all probe sequences, the same level of multiplexing can be achieved albeit at a lower cost with lower background noises using less total fluorophore reporters.

For example, rather than adding red at 33%, red at 66%, and red at 100%, and similar for blue, orange, and yellow, totaling 200% concentrations of fluorophores of each color, dramatically increasing background levels, 33% concentrations for all colors can be added, totaling only 100% in each color band. Multiplexing is then enabled by adding non-labeled probes at 0%, 33%, and 66% to achieve competition based temporal shifts. Thus, multiplexing ability across several colors can be significantly increased while keeping total background fluorescence levels low.

In an alternative embodiment, 33% concentration TaqMan labeled probes combined with primer concentration restricted to 33%, 66%, and 100% concentrations can be used. Multiplexing is achieved through the use of intercalating dyes to indicate total DNA amplification concentrations within all digital reactors. This is applicable across color bands and helps reduce costs while reducing background fluorescence levels in each color spectra. Even in the green color spectra where the intercalating dyes are detected for every single reaction, the background level is not increased and remains constant at 100% regardless of the additive multiplexing techniques it is combined with. This provides greater degrees of primer/probe differentiation using color and intensity cues, induced by both specific and non-specific reporters and amplification schemes.

Example illustration is as follows:
Probe 1, 33% red 33% primer
Probe 2, 33% red, 66% primer
Probe 3, 33% red, 100% primer
Probe 4, 33% blue, 33% primer
Probe 5, 33% blue, 66% primer
Probe 6, 33% blue, 100% primer

Probe 7, 33% orange, 33% primer
Probe 8, 33°7˜orange, 66% primer
Probe 9, 33% orange, 100% primer
Probe 10, 33% yellow, 33% primer
Probe 11, 33% yellow, 66% primer
Probe 12, 33% yellow, 100% primer
Probe 13, 33% primer
Probe 14, 66% primer
Probe 15, 100% primer
   Total concentrations:
   Red=100%, blue=100%, orange=100%, yellow—100%, intercalating dye (green)—100%.

Now 15-plex amplification is achieved, using end-point, or real-time detection if desired, with no more than 100% total reporter concentrations in any given spectral band. This is true even in the green spectrum which is used to help identify over 15 primer/probe pairs. Further additions of multiplexed probes which utilize information in the green spectra will not increase the green background fluorescence levels. Such powerful and cost efficient digital multiplexing ability at such low background signal levels cannot be achieved using the techniques put forth in the previous digital multiplexing embodiments.

According to a still further embodiment, melting-curve analysis may be performed of amplified DNA contents using intercalating dyes to differentiate between DNA strands of one annealing temperature vs. another temperature.

A still further alternate embodiment is to use TaqMan primer/probe sets labeled with green reporters, and combine it with non TaqMan probe intercalating dyes of the same green colored wavelength to differentiate a greater number of primer, and primer/probe pairs using only a single color spectrum. This process allows greater multiplexing ability using a single color filter, and can be analyzed using real-time measurements or end-point intensity measurements alone. For example, three distinct TaqMan labeled probes can be used with both primer concentrations and reporters limited at 33%, 66%, and 100% intensity concentrations, respectively. In the same reaction, three non TaqMan primer pairs can be utilized which are limited in primer concentration to yield 33%, 66%, and 100% intensity intercalating dyes. Three additional TaqMan probe labeled combinations can be encoded using (1) 66% primer limited and 33% TaqMan probe limited concentrations, and (2 and 3) 100% primer limited with 33% and 66% TaqMan probe label limited concentrations. Using either real-time or end-point analysis alone, 9 different primer/probe pairs on a single color band can be differentiated with relatively low background noise signals.

The contribution of TaqMan labeled probes to total fluorescence intensity is measured at a high temperature, e.g., 90 C., where DNA strands are single stranded and intercalating dye intensity is very low. Here, which digital reactors containing 33%, 66%, and 100% TaqMan probe limited fluorescence concentrations may be determined. Then, the total intensity of both the TaqMan probe and intercalating dye together are measured at a lower temperature when DNA strands are annealed together. From this, the differences in intensity resulting from temperature may be corrected, and the percentage of the amplified signal contributed by intercalating dye concentrations and whether the amplified signal results from 33%, 66%, or 100% primer limited concentrations can be interpolated. From this information the combinations of primer limited probes, TaqMan limited probes, and primer and TaqMan limited probe concentrations can be reconstituted to the overall intensity levels of the digital reactors. Now a 12-plex reaction can be identified on a single color band using end-point analysis of each reactor. Other variations can be performed which incorporate real-time analysis of fluorescence intensities to utilize temporal shifts and competition based fluorescence signatures in a single color band. Other variations can be used by adding TaqMan probes and intercalating dyes in prime concentrations so that their unique combinations can yield a greater variety of intensity encodings assuming the fluorescence detection sensitivity can accommodate such a scheme. Any variety of these approaches may be used, alone or in combination with each other, to enable extremely versatile and cost affordable multiplexing techniques in the digital PCR regime with lower probe reporter concentrations and fewer spectral bands and even non TaqMan probe reporters.

1. A method for amplifying a DNA sequence with a polymerase chain reaction (PCR), the method comprising:
discretizing a volume of concentration comprising a plurality of DNA samples into a plurality of reactor volumes such that a presence of a DNA sample in a reactor volume is digitized;
performing a multiplex polymerase chain reaction (PCR) process in the plurality of reactor volumes to amplify a plurality of DNA sequences in each reactor volume;
emitting fluorescences in a plurality of spectral wavelengths when an DNA sequence of the plurality of DNA sequences is amplified; and
identifying the DNA sequences amplified according to the emitted fluorescences,
wherein the volume of concentration comprises a plurality of primers, each of the plurality of primers corresponding to a DNA sequence of the plurality of DNA sequences and comprising a fluorescent reporter configured to emit fluorescence in a spectral wavelength of the plurality of spectral wavelengths when the corresponding DNA sequence is amplified.

2. The method according to claim 2, wherein emitting fluorescence in a plurality of spectral wavelengths when a DNA sequence is amplified comprises emitting fluorescences from the fluorescent reporters comprised in the plurality of primers in a color combination of a plurality of color combinations, the color combination comprising a combination of spectral wavelengths of the plurality of spectral wavelengths.

3. The method according to claim 2, wherein the color combination corresponds uniquely to a particular DNA sequence of the plurality of DNA sequences.

4. The method according to claim 3, wherein identifying the DNA sequences amplified according to the emitted fluorescences comprises:
determining the color combinations comprising the emitted fluorescences; and
identifying the particular DNA sequences corresponding to the emitted color combinations.

5. The method according to claim 1, wherein each fluorescent reporter of the plurality of fluorescent reporters is further configured to emit fluorescence in a spectral wavelength at a pre-defined intensity.

6. The method according to claim 5, wherein the spectral wavelength and the pre-defined intensity uniquely correspond to the DNA sequence.

7. The method according to claim 6, wherein emitting fluorescences in a plurality of spectral wavelengths when a DNA sequence is amplified comprises emitting fluorescence in at least one of: 1) a different spectral wavelength; and 2) at a different pre-defined intensity, for each of the plurality of DNA sequences.

8. The method according to claim 5, wherein the pre-defined intensity in the fluorescence in a spectral wavelength emitted by the fluorescent reporter corresponds to a concentration of the fluorescent reporter comprised in the primer.

9. The method according to claim 5, wherein identifying the DNA sequences amplified according to the emitted fluorescences comprises:
determining the intensities and spectral wavelengths comprising the emitted fluorescences; and
identifying the particular DNA sequences corresponding to the emitted intensities and spectral wavelengths.

10. The method according to claim 1, wherein emitting fluorescence in a plurality of spectral wavelengths when a DNA sequence is amplified comprises emitting fluorescences from the fluorescent reporters comprised in the plurality of primers in a color combination of a plurality of color combinations at an intensity of a plurality of pre-defined intensities.

11. The method according to claim 10, wherein a color combination and pre-defined intensity corresponds uniquely to a particular DNA sequence.

12. The method according to claim 11, wherein identifying the DNA sequences amplified according to the emitted fluorescences comprises:
determining the color combinations and intensities comprising the emitted fluorescences; and
identifying the particular DNA sequences corresponding to the color combinations and intensities.

13. The method according to claim 1, wherein performing a multiplex polymerase chain reaction (PCR) process in the plurality of reactor volumes to amplify a plurality of DNA sequences in each reactor volume comprises performing a plurality of thermocycles.

14. The method according to claim 13, wherein each fluorescent reporter of the plurality of fluorescent reporters is further configured to emit fluorescence in a spectral wavelength after a pre-defined number of thermocycles have elapsed.

15. The method according to claim 14, wherein the pre-defined number of thermocycles after which a fluorescent reporter emits fluorescence corresponds uniquely to a particular DNA sequence of the plurality of DNA sequences.

16. The method according to claim 15, wherein the volume of concentration further comprises a second plurality of primers corresponding to a plurality of DNA sequences, the second plurality of primers not comprising fluorescent reporters.

17. The method according to claim 16, wherein the pre-defined number of thermocycles after which a fluorescent reporter emits fluorescence for a particular DNA sequence corresponds to the ratio of primers of the plurality of primers to primers of the second plurality of primers.

18. The method according to claim 14, wherein the plurality of primers comprise primers configured bind to DNA sequences at varying efficiencies.

19. The method according to claim 14, wherein a primer of the plurality of primers configured to bind to a DNA sequence with a higher efficiency will emit fluorescence after a lower number of thermocycles has elapsed than a primer configured to bind to a DNA sequence at a lower efficiency.

20. The method according to claim 14, wherein the plurality of primers comprise primers that are configured to bind to a DNA sequence at varying temperatures in the plurality of reactor volumes.

21. The method according to claim 20, wherein the temperature in the plurality of reactor volumes is cycled through a pre-defined range during a thermocycle of the plurality of thermocycles.

22. The method according to claim 14, wherein identifying the DNA sequences amplified according to the emitted fluorescences comprises:
determining the numbers of thermocycles performed by the multiplex PCR process prior to emitting fluorescences; and
identifying the particular DNA sequences corresponding to the numbers of thermocycles.

23. The method according to claim 1, wherein the fluorescent reporter comprises an intercalating dye.

24. The method according to claim 1, wherein the fluorescent reporter comprises a probe comprising a fluorophore and a quencher.

25. The method according to claim 24, wherein the problem comprises a TaqMan probe.

26. A system for performing multiplex polymerase chain reaction (PCR), the system comprising:
a plurality of DNA samples;
a volume of concentration comprising the plurality of DNA samples;
a plurality of reactor volumes comprising a discretized distribution of the volume of concentration, each reactor volume being configured such that a presence of a DNA sample is digitized; and
a thermal cycler coupled to the plurality of reactor volumes and operable to configure a temperature in the plurality of reactor volumes,
wherein the volume of concentration comprises a plurality of primers, each of the plurality of primers is configured to bind to, and amplify a DNA sequence of the plurality of DNA sequences in conjunction with a DNA template,
wherein the plurality of primers comprises a plurality of fluorescent reporters configured to emit fluorescence in a plurality of spectral wavelengths when a DNA sequence corresponding to a primer of the plurality of primers is amplified.

27. The system according to claim 26, further comprising a filter for detecting fluorescence emitted by a fluorescent reporter of the plurality of fluorescent reporters.

28. The system according to claim 26, wherein the plurality of fluorescent reporters comprised in the plurality of primers are configured to emit fluorescence in a color combination of a plurality of color combinations, the color combination comprising a combination of spectral wavelengths of the plurality of spectral wavelengths.

29. The system according to claim 28, wherein the color combination corresponds uniquely to a particular DNA sequence of the plurality of DNA sequences.

30. The system according to claim 29, wherein the DNA sequences amplified during a PCR process are identified based on the emitted color combinations.

31. The system according to claim 26, wherein each fluorescent reporter of the plurality of fluorescent reporters is further configured to emit fluorescence in a spectral wavelength at a pre-defined intensity.

32. The system according to claim 31, wherein the spectral wavelength and the pre-defined intensity uniquely correspond to the DNA sequence.

33. The system according to claim 32, wherein the plurality of fluorescent reporters is configured to emit fluorescence in at least one of: 1) a different spectral wavelength; and 2) at a different pre-defined intensity, for each of the plurality of DNA sequences.

34. The system according to claim 31, wherein the pre-defined intensity in the fluorescence in a spectral wavelength emitted by the fluorescent reporter corresponds to a concentration of the fluorescent reporter comprised in the primer.

35. The system according to claim 31, wherein the DNA sequences amplified during a PCR process are identified based on the emitted intensities and spectral wavelengths.

36. The system according to claim 26, wherein the plurality of fluorescent reporters comprised in the plurality of primers are configured to emit fluorescence in a color combination of a plurality of color combinations at an intensity of a plurality of pre-defined intensities.

37. The system according to claim 36, wherein a color combination and pre-defined intensity corresponds uniquely to a particular DNA sequence.

38. The system according to claim 37, wherein the DNA sequences amplified during a PCR process are identified based on the emitted color combinations and intensities.

39. The system according to claim 26, wherein the thermal cycler is configured to cycle the temperature in the plurality of reactor volumes in a plurality of thermocycles.

40. The system according to claim 39, wherein each fluorescent reporter of the plurality of fluorescent reporters is further configured to emit fluorescence in a spectral wavelength after a pre-defined number of thermocycles have elapsed.

41. The system according to claim 40, wherein the pre-defined number of thermocycles after which a fluorescent reporter emits fluorescence corresponds uniquely to a particular DNA sequence of the plurality of DNA sequences.

42. The system according to claim 41, wherein the volume of concentration further comprises a second plurality of primers corresponding to a plurality of DNA sequences and that do not comprise fluorescent reporters.

43. The system according to claim 42, wherein the pre-defined number of thermocycles after which a fluorescent reporter emits fluorescence for a particular DNA sequence corresponds to the ratio of primers from the plurality of primers to the primers from the second plurality of primers.

44. The system according to claim 40, wherein the plurality of primers comprise primers configured bind to DNA sequences at varying efficiencies.

45. The system according to claim 40, wherein a primer of the plurality of primers configured to bind to a DNA sequence with a higher efficiency will emit fluorescence after a lower number of thermocycles has elapsed than a primer configured to bind to a DNA sequence at a lower efficiency.

46. The system according to claim 40, wherein the plurality of primers comprise primers that are configured to bind to a DNA sequence at varying temperatures in the plurality of reactor volumes.

47. The system according to claim 46, wherein the temperature in the plurality of reactor volumes is cycled through a pre-defined range during a thermocycle of the plurality of thermocycles.

48. The system according to claim 40, wherein the DNA sequences amplified during a PCR process are identified based on the numbers of thermocycles elapsed before fluorescences are emitted.

49. The system according to claim 26, wherein the fluorescent reporter comprises an intercalating dye.

50. The system according to claim 26, wherein the fluorescent reporter comprises a probe comprising a fluorophore and a quencher.

51. The system according to claim 50, wherein the problem comprises a TaqMan probe.

52. The system according to claim 26, wherein the plurality of reactor volumes comprises one million reactor volumes.
